(19) United States
(12) Patent Application Publication
(10) Pub. No.: US 2010/0179646 A1
(43) Pub. Date: Jul. 15, 2010

(54) GLUCOSE OXIDASE TECHNIQUES

(75) Inventors: Yossi Gross, Moshav Mazor (IL); Hadar Better, Tel Aviv (IL)

Correspondence Address: RATNERPRESTIA, P.O. BOX 980, VALLEY FORGE, PA, 19482, US

(73) Assignee: Rainbow Medical Ltd., Herzliya (IL)

(21) Appl. No.: 12/685,111

(22) Filed: Jan. 11, 2010

Related U.S. Application Data
(60) Provisional application No. 61/204,683, filed on Jan. 9, 2009.
(60) Provisional application No. 61/274,633, filed on Aug. 18, 2009.

(51) Int. Cl. A61F 2/24; A61F 2/00; A61K 38/44; A61L 17/00; A61M 25/00; A61F 2/82

(52) U.S. Cl. 623/1.42; 424/423; 424/94.4; 606/228; 604/265; 623/2.1

(57) ABSTRACT

Apparatus and methods are described including an implantable medical device for implanting in contact with a portion of a subject's body. Glucose oxidase, having a mass thereof, is coupled to the medical device. The medical device does not have coupled thereto a mass of glucose that exceeds 0.01 percent of the mass of the glucose oxidase. Other embodiments are also described.

### CROSS-REFERENCES TO RELATED APPLICATIONS

The present application claims the benefit of U.S. Provisional Patent Application 61/204,683 to Gross, filed Jan. 9, 2009, entitled “Glucose oxidase for medical devices,” and U.S. Provisional Patent Application 61/274,633 to Gross, filed Aug. 18, 2009, entitled “Glucose oxidase techniques.” Both of the aforementioned applications are incorporated herein by reference.

### FIELD OF EMBODIMENTS OF THE INVENTION

Some applications of the present invention generally relate to medical apparatus. Specifically, some applications relate to techniques for use with glucose oxidase.

### BACKGROUND

The glucose oxidase enzyme (GOx) binds to beta-D-glucose (an isomer of the six-carbon sugar glucose) and aids in breaking the sugar down into its metabolites. The GOx-catalyzed reaction results in the reduction of oxygen to hydrogen peroxide.

The GOx enzyme is commonly used in biosensors to detect levels of glucose by keeping track of the number of electrons passed through the enzyme by connecting it to an electrode and measuring the resulting charge. When produced commercially for this application, it is often extracted from aspergillus niger.

GOx found in honey and acts as a natural preservative. GOx at the surface of the honey reduces atmospheric oxygen to hydrogen peroxide, which acts as an antimicrobial barrier.

Archimed™ (UK) manufactures OXYZYME™ and IODOZYME™ wound dressings.

Nexcare™ (MN, USA) manufactures Nexcare™ No Sting Liquid Bandage.

Laclede, Inc. (CA, USA) manufactures a range of products that contain glucose oxidase, under the trade name Biotene®.

Dexcel® Pharma (Ohr Akiva, Israel) manufactures chips of chlorhexidine gluconate, for insertion into periodontal pockets, under the trade name PerioChip®.

The following references may be of interest:
US 2006/0034816 to Davis
US 2006/0275350 to Davis
US 2006/0276844 to Alon
US 2006/0281165 to Davis
US 2007/0112174 to Shiba
US 2007/0148117 to Davis
US 2007/0190122 to Davis
US 2009/0081279 to Jezek
US 2009/0169600 to Davis
U.S. Pat. No. 4,150,113 to Hoogendoorn
U.S. Pat. No. 4,576,817 to Montgomery
U.S. Pat. No. 4,617,190 to Montgomery
U.S. Pat. No. 5,849,041 to Kunz
U.S. Pat. No. 5,874,109 to Ducheyne
U.S. Pat. No. 6,033,662 to Allen
U.S. Pat. No. 6,214,339 to Pellico
U.S. Pat. No. 6,482,309 to Green
U.S. Pat. No. 6,696,058 to Pellico
U.S. Pat. No. 6,838,050 to Green
U.S. Pat. No. 6,939,569 to Green
WO 97/026908 to Claesson
WO 01/045762 to Sollie
WO 04/064881 to Davis
WO 06/133523 to Van den Plas
WO 07/128,985 to Austin
WO 08/012,107 to Van den Plas
WO 08/084,477 to Gross
WO 09/019,498 to Jezek
WO 09/019,499 to Jezek
“Effects of diabetes on the osseointegration of dental implants,” by Valero, Med Oral Patol Oral Cir Bucal 2007; 12:E38-43
“Enzymatic Activity of Glucose Oxidase Encapsulated in Transparent Glass by the Sol-Gel Method,” by Yamanaka, Chemistry of Materials, VOLUME 4, NUMBER 3, MAY/JUNE 1992
“Eradication of Interleukin 5-transfected J558L Plasmacytomas in Mice by Hydrogen Peroxide-generating Stealth Liposomes,” by Samoszuk, Cancer Research 56, 87-90, Jan. 1, 1996
“Wound healing with honey—a randomised controlled trial,” by Rode, May 2007, SAMJ Vol. 97, No. 5

### SUMMARY OF EMBODIMENTS

For some applications of the present invention, GOx is administered to a subject and reacts with native glucose that is present in the subject's body. For some applications, no glucose is administered to the subject, or glucose is administered to the subject, the glucose having a mass thereof that does not exceed 0.01 percent of the mass of the glucose oxidase.

For some applications, the glucose oxidase is coupled to an implanted medical device. Typically, the medical device generates hydrogen peroxide in the vicinity of the device by facilitating a reaction of the glucose oxidase with native glucose that is present in tissue of the subject in the vicinity. Further typically, the medical device reduces infection in a vicinity of the portion by facilitating a reaction of the glucose oxidase with native glucose that is present in tissue of the subject in the vicinity.

For some applications, a sensor detects a parameter of the vicinity of the medical device, and the device releases the glucose oxidase responsively to the detected parameter. For example, a pH sensor may detect the pH of the vicinity. For some applications, the pH sensor comprises a pH sensitive coating, which dissolves and releases the glucose oxidase in response to the pH of the vicinity passing a threshold. Alternatively or additionally, the element releases the glucose oxidase in response to the pH of the vicinity passing a threshold in a first direction, and stops releasing the glucose oxidase in response to the pH passing the threshold in a second direction.

For some applications, the medical element is configured to release the mass of glucose oxidase from the medical element over a period of more than a week, for example, more than a month, or more than six months.

For some applications, a medical device is placed inside a subject's body. Particles (e.g., nanoparticles) are subsequently administered to the subject, the particles (a) being couplable to the medical device and (b) having one or more glucose oxidase molecules coupled to each of the particles.

For some applications, glucose oxidase is administered to a subject who is susceptible to blood clotting. The blood clotting is reduced by the glucose oxidase reacting with native glucose that is present in the subject's blood.

For some applications, a substance that contains glucose oxidase is administered to a subject's intestine. For some application, the substance (a) reduces a level of glucose in the intestine, (b) reduces a level of absorption by the intestine, and/or (c) reduces a level of infection in the intestine by facilitating a reaction between the glucose oxidase and native glucose that is present in the intestine. Alternatively or additionally, the substance includes glucose, and the glucose oxidase and the glucose react inside the intestine, causing (a) a reduction in the absorption of the intestine, and/or (b) a reduction in a level of infection in the intestine. For some applications, the substance is administered in response to identifying that the subject is suffering from inflammatory bowel disease, diabetes, and/or obesity.

It is noted that the term “native glucose” as used in the present application is to be understood to mean glucose that is naturally (i.e., without any intervention) present in a portion of the subject's body. For example, glucose in blood, tissue, or glucose from ingested matter that is present in the body. “Native glucose” does not include, for example, glucose that is incorporated in a medical device.

There is therefore provided, in accordance with some applications of the present invention, apparatus, including:

an implantable medical device for implanting in contact with a portion of a subject's body; and

glucose oxidase, having a mass thereof, coupled to the medical device,

the medical device not having coupled thereto a mass of glucose that exceeds 0.01 percent of the mass of the glucose oxidase.

For some applications, the medical device includes nanoparticles on a surface thereof the glucose oxidase being coupled to the nanoparticles.

For some applications, the glucose oxidase includes nanoparticles of glucose oxidase.

For some applications, the medical device is configured to generate hydrogen peroxide in a vicinity of the portion by facilitating a reaction of the glucose oxidase with native glucose that is present in tissue of the subject in the vicinity.

For some applications, the medical device is configured to reduce infection in a vicinity of the portion by facilitating a reaction of the glucose oxidase with native glucose that is present in tissue of the subject in the vicinity.

For some applications, the medical device includes a suture.

For some applications, the medical device includes a catheter.

For some applications, the medical device includes a medical device selected from the group consisting of: a mesh, and a membrane.

For some applications, the medical device includes a birth control implant.

For some applications, the medical device includes an injectable filler.

For some applications, the medical device is configured to reduce blood coagulation in a vicinity of the portion by facilitating a reaction of the glucose oxidase with native glucose that is present in tissue of the subject in the vicinity.

For some applications, the device includes a stent.

For some applications, the medical device is configured to reduce fibrotic encapsulation of the device by facilitating a reaction of the glucose oxidase with native glucose that is present in tissue of the subject in a vicinity of the portion.

For some applications, the device includes a stent.

For some applications, the apparatus further includes a sensor configured to detect a parameter of the vicinity, the device is configured to release the glucose oxidase responsively to the detected parameter.

For some applications, the sensor includes a pH sensor configured to detect a pH of the vicinity.

For some applications, the pH sensor includes a pH sensitive coating configured to dissolve and release the glucose oxidase in response to the pH of the vicinity passing a threshold.

For some applications, the device is configured to release the glucose oxidase in response to the pH of the vicinity passing a starting threshold in a first direction, and to stop releasing the glucose oxidase in response to the pH passing a stopping threshold in a second direction opposite to the first direction.

For some applications, the medical device is configured to release the mass of glucose oxidase from the medical device over a period of more than a week.

For some applications, the medical device is configured to release the mass of glucose oxidase from the medical device over a period of more than a month.

For some applications, the medical device is configured to release the mass of glucose oxidase from the medical device over a period of more than six months.

For some applications, the medical device includes a heart valve configured to be implanted in a heart of the subject.

For some applications, the heart valve includes a reservoir configured to store the mass of glucose oxidase, to release the mass of glucose oxidase over a period of more than a month.

For some applications, the medical device includes a bone generation facilitator configured to facilitate bone generation in a region of the subject's body by being placed in the region.

For some applications, the glucose oxidase is configured to facilitate the bone generation.

For some applications, the medical device includes a medical device selected from the group consisting of: bone graft, bone graft powder, a paste, an injected material, and a metal.

For some applications, the medical device includes a membrane configured to facilitate bone generation in the region of the subject's body by being placed in the region.

For some applications, the membrane includes a collagen barrier.

For some applications, the membrane includes a polymer selected from the group consisting of: polytetrafluoroethylene and expanded polytetrafluoroethylene.

There is further provided, in accordance with some applications of the present invention, apparatus, including:

an implantable medical device for placing inside a subject's body;

particles couplable to the medical device inside the subject's body by being administered to the subject's body; and

one or more glucose oxidase molecules coupled to each of the particles.

For some applications, the apparatus further includes a solution containing the particles and the glucose oxidase molecules.

For some applications, the particles include nanoparticles.

For some applications, each of the particles includes a ligand, and the medical device includes receptors possessing affinity for the ligand.

For some applications, the medical device includes a ligand, and each of the particles includes a receptor possessing affinity for the ligand.

For some applications, each of the particles includes a magnetic material, the medical device includes a magnetic material, and the particles are couplable to the medical device via the magnetic materials.

For some applications, the medical device includes an antigen, and each particle includes an antibody configured to couple to the antigen of the medical device.

For some applications, the medical device includes an antibody, and each particle includes an antigen configured to couple to the antibody of the medical device.

For some applications, the medical device includes an intramedullary nail.

There is additionally provided, in accordance with some applications of the present invention, a composition of matter, including:

a varnish; and

glucose oxidase disposed within the varnish.

For some applications, the varnish includes nail varnish.

For some applications, the varnish is configured to be applied to a bone of a subject.

For some applications, the varnish is configured to be applied to cartilage of a subject.

For some applications, the varnish is configured to be applied to skin of a subject.

There is further provided, in accordance with some applications of the present invention, a method, including:

providing an implantable medical device for implanting in contact with a portion of a subject's body; and

coupling to the device glucose oxidase having a mass thereof, the device not having coupled thereto glucose having a mass thereof that exceeds 0.01 percent of the mass of the glucose oxidase.

There is further provided, in accordance with some applications of the present invention, a method, including:

identifying a nail of a subject suffering from onychomycosis; and

applying to the nail varnish that contains glucose oxidase.

There is additionally provided, in accordance with some applications of the present invention, a method, including:

identifying that a medical device inside a subject's body has less than a desired amount of glucose oxidase; and

in response thereto, coupling to the medical device particles that include glucose oxidase, by injecting the particles into the subject's body.

There is further provided, in accordance with some applications of the present invention, a method, including:

identifying a subject, at least a portion of a body of which subject is in a hyper-coagulation state; and

in response to the identifying, reducing blood clotting of the portion by facilitating a reaction of glucose oxidase with native glucose that is present in blood of the subject, by administering glucose oxidase to the portion.

There is additionally provided, in accordance with some applications of the present invention, apparatus, including:

a substance configured to be administered to an intestine of a subject; and

glucose oxidase, disposed within the substance.

For some applications, the glucose oxidase has a mass thereof, and the substance does not have disposed therein glucose having a mass thereof that exceeds 0.01 percent of the mass of the glucose oxidase.

For some applications, the substance includes a substance selected from the group consisting of: a capsule, a liquid, a syrup, a powder and a gel.

For some applications, the substance is configured to be administered to the intestine by being sprayed from an endoscope.

For some applications, the substance is configured to reduce a presence of glucose in the intestine by facilitating a reaction of the glucose oxidase with native glucose that is present in the intestine.

For some applications, the substance is configured to reduce absorption of the intestine by facilitating a reaction of the glucose oxidase with native glucose that is present in the intestine.

For some applications, the apparatus further includes glucose disposed within the substance, and the substance is configured to reduce absorption of the intestine by facilitating a reaction of the glucose oxidase with the glucose, while the substance is inside the intestine.

For some applications, the substance is configured to reduce infection in the intestine by facilitating a reaction of the glucose oxidase with native glucose that is present in the intestine.

For some applications, the apparatus further includes glucose disposed within the substance, and the substance is configured to reduce infection in the intestine by facilitating a reaction of the glucose oxidase with the glucose, while the substance is inside the intestine.

For some applications, the substance is configured to reduce inflammation in the intestine by facilitating a reaction of the glucose oxidase with native glucose that is present in the intestine.

For some applications, the apparatus further includes glucose disposed within the substance, and the substance is configured to reduce inflammation in the intestine by facilitating a reaction of the glucose oxidase with the glucose, while the substance is inside the intestine.

For some applications, the substance is configured to reduce a residue that is bound to a surface of the intestine by facilitating a reaction of the glucose oxidase with native glucose that is present in the intestine.

For some applications, the apparatus further includes glucose disposed within the substance, and the substance is configured to reduce a residue that is bound to a surface of the intestine by facilitating a reaction of the glucose oxidase with the glucose, while the substance is inside the intestine.

There is additionally provided, in accordance with some applications of the present invention, a method including:

providing a substance that includes glucose oxidase; and

administering the substance to an intestine of the subject.

For some applications, the method further includes identifying that the subject suffers from inflammatory bowel disease, and administering the substance to the intestine includes, in response to the identifying, reducing a level of inflammation in the subject's intestine by facilitating a reaction of the glucose oxidase with native glucose that is present in the intestine, by administering the substance to the subject's intestine.

For some applications, the method further includes identifying that the subject suffers from inflammatory bowel disease,

providing the substance includes providing a substance that includes glucose oxidase and glucose, and

administering the substance to the intestine includes, in response to the identifying, reducing a level of inflammation in the subject's intestine by facilitating a reaction of the glucose oxidase with the glucose, by administering the substance to the subject's intestine.

For some applications, administering the substance to the intestine includes reducing a presence of glucose in the intestine by facilitating a reaction of the glucose oxidase with native glucose that is present in the intestine, by administering the substance to the subject's intestine.

For some applications, the method further includes identifying that the subject is obese, and administering the substance to the intestine includes administering the substance to the intestine in response to identifying that the subject is obese.

For some applications, the method further includes identifying that the subject is diabetic, and administering the substance to the intestine includes administering the substance to the intestine in response to identifying that the subject is diabetic.

There is additionally provided, in accordance with some applications of the present invention, apparatus, including:

a powder composition, in dry form, including:
glucose oxidase; and
glucose.

For some applications, the powder composition includes talcum powder.

For some applications, the apparatus further includes a hair dye solution, and the powder is configured to be added to the hair dye solution.

For some applications, the powder composition is configured to be administered to skin of a subject, and the powder is configured, by contacting a bodily fluid of the subject, to generate hydrogen peroxide on the skin by facilitating a reaction of the glucose oxidase with the glucose.

For some applications, the powder composition is configured to reduce an odor of the skin by facilitating the reaction.

There is additionally provided, in accordance with some applications of the present invention, apparatus, including:

a medical element, for placing in contact with a portion of a subject's body;

glucose oxidase, coupled to the element; and

glucose coupled to the element,

before the element is placed in contact with the portion, the glucose and the glucose oxidase not being in direct contact with each other, and the element being configured to bring the glucose and glucose oxidase into direct contact with each other by being placed in contact with the portion.

For some applications, the element includes first and second chambers, at least some of the glucose oxidase is disposed inside the first chamber and at least some of the glucose is disposed inside the second chamber, and the first and second chambers are configured to keep the glucose and glucose oxidase separate from each other before the element is placed in contact with the portion.

For some applications, the element includes chewing gum configured to bring the glucose and glucose oxidase into direct contact with each other by being placed inside the subject's mouth.

For some applications:

the element includes an absorbent article configured to be placed in contact with a portion of a body of a subject from which a bodily fluid is released,

the glucose oxidase includes glucose oxidase in dry form, coupled to the article,

the glucose includes glucose in dry form, coupled to the article, and

the article is configured, by contacting the bodily fluid, to facilitate a reaction of the glucose oxidase with the glucose by bringing the glucose and glucose oxidase in direct contact with each other.

For some applications, the article includes an article selected from the group consisting of: a diaper, a tampon, a sanitary towel, and a bandage.

For some applications, the article is configured to reduce infection in a vicinity of the article by facilitating the reaction of the glucose oxidase with the glucose.

For some applications, the article is configured to reduce an odor in a vicinity of the article by facilitating the reaction of the glucose oxidase with the glucose.

There is additionally provided, in accordance with some applications of the present invention, apparatus, including:

a pellet configured to be injected into a portion of a subject's body, the pellet including glucose oxidase.

For some applications, the pellet has a volume of 0.01 cc to 0.1 cc.

For some applications, the pellet is configured to reduce infection in the portion by facilitating a reaction of the glucose oxidase with native glucose that is present in tissue of the subject in the portion.

For some applications, the pellet further includes glucose, and the pellet is configured to reduce infection in the portion by facilitating a reaction of the glucose oxidase with the glucose by contacting a fluid in the portion.

There is additionally provided, in accordance with some applications of the present invention, a method, including:

identifying a subject, a portion of a body of which subject is suffering from a local infection; and

injecting glucose oxidase into the portion in response to the identifying.

For some applications, injecting the glucose oxidase includes injecting a solution that contains glucose oxidase.

For some applications, injecting the glucose oxidase includes inserting a needle into the portion, glucose oxidase being disposed on an outer surface of the needle.

For some applications, the method further includes administering glucose to the portion.

There is additionally provided, in accordance with some applications of the present invention, a method, including:

providing an implantable medical device for implanting in contact with a portion of a subject's body, the device having coupled thereto glucose oxidase having a mass thereof, the device not having coupled thereto glucose having a mass thereof that exceeds 0.01 percent of the mass of the glucose oxidase; and

implanting the device in contact with the portion.

For some applications, providing the device includes coupling nanoparticles to the device, while the device is inside the subject's body, by injecting the nanoparticles into the subject's body, the glucose oxidase being coupled to the nanoparticles, and the nanoparticles being couplable to the device.

For some applications, providing the device includes coupling nanoparticles of glucose oxidase to the device, while the device is inside the subject's body, by injecting the nanoparticles into the subject's body, the nanoparticles being couplable to the device.

There is additionally provided, in accordance with some applications of the present invention, a method including:

providing a substance configured to be administered to an intestine of a subject; and

including within the substance glucose oxidase.

The present invention will be more fully understood from the following detailed description of embodiments thereof, taken together with the drawings, in which:

### BRIEF DESCRIPTION OF THE DRAWINGS

FIG. 1 is a schematic illustration of a medical device that is coated with glucose oxidase, in accordance with some applications of the present invention;

FIG. 2 is a schematic illustration of a medical device having coupled thereto particles that contain glucose oxidase, in accordance with some applications of the present invention;

FIG. 3 is a schematic illustration of a piece of a medical element that contains, in separate chambers, glucose oxidase and glucose, in accordance with some applications of the present invention;

FIG. 4 is a schematic illustration of an absorbent article containing dry particles of glucose oxidase and glucose, in accordance with some applications of the present invention;

FIG. 5 is a graph showing experimental results that demonstrate the relationship between GOx concentration and GOx activity;

FIGS. 6A and 6B are graphs showing experimental results that demonstrate the relationship between glucose concentration and GOx activity;

FIG. 7 is a graph showing experimental results that demonstrate the relationship between pH and GOx activity;

FIGS. 8A and 8B are graphs showing experimental results that demonstrate the minimal inhibitory concentration of GOx as an antibacterial agent;

FIG. 9 is a set of graphs showing experimental results that demonstrate the sustainability of GOx as an antibacterial agent; and

FIGS. 10A and 10B are graphs showing experimental results that demonstrate the minimal inhibitory concentration of Triclosan as an antibacterial agent.

### DETAILED DESCRIPTION OF EMBODIMENTS

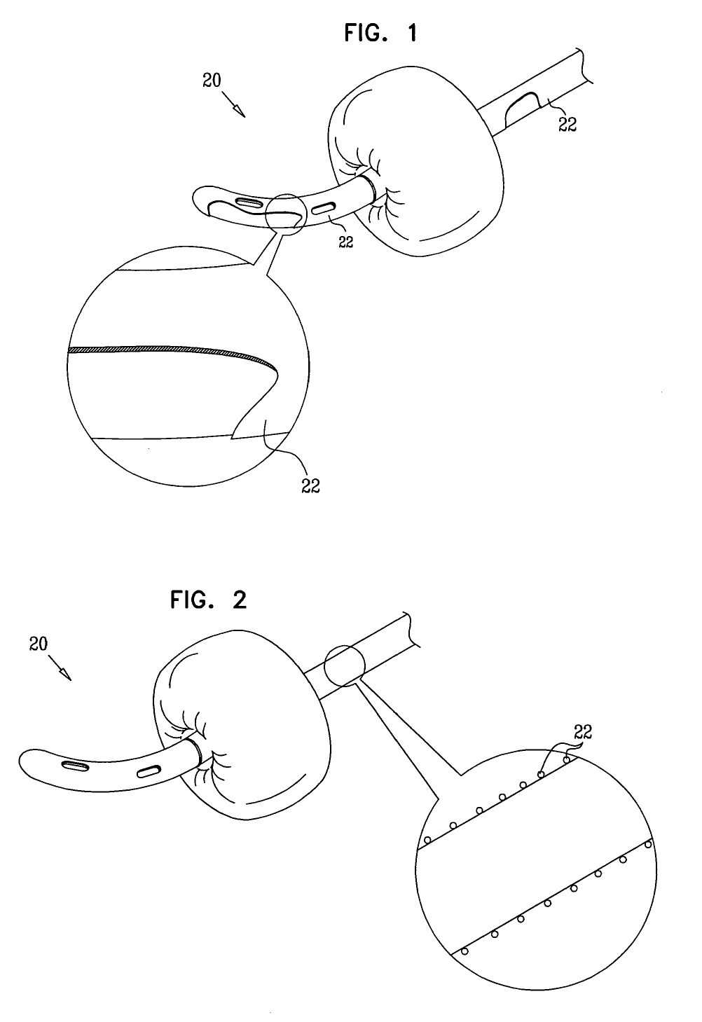

Reference is now made to FIG. 1, which is a schematic illustration of a medical device 20 that is coated with glucose oxidase 22, in accordance with some applications of the present invention. Typically, the medical device does not have glucose coupled thereto, or does not have coupled thereto a substantial amount of glucose, e.g., a mass of glucose that exceeds 0.01 percent of the mass of the glucose oxidase that is coupled to the medical device.

For some applications, as shown, the device is a catheter (e.g., a balloon catheter), as is generally known in the art. Alternatively or additionally, the device is a suture, a mesh, a membrane, a birth control implant, an injectable filler (as is used in plastic surgery), a stent, an artificial heart valve, an intramedullary nail, and/or another type of implant that is known in the art.

Typically, device 20 is implanted in a portion of a subject's body, and glucose oxidase 22 generates hydrogen peroxide in a vicinity of the portion by reacting with native glucose that is present in tissue of the subject in the vicinity. For some applications, the glucose oxidase reduces infection in the vicinity by reacting with the native glucose. For some applications, the glucose oxidase reduces blood coagulation in the vicinity by reacting with the native glucose. Alternatively or additionally, the glucose oxidase reduces fibrotic encapsulation of the device (for example, of a stent) by reacting with the native glucose.

For some applications, a sensor detects a parameter of the vicinity of the portion in which device 20 is implanted. Glucose oxidase 22 is released responsively to the detected parameter. For example, a control unit (not shown) may be electrically coupled to the device and control the release of the glucose oxidase in response to the detected parameter.

For some applications, the device includes a pH-sensor. For example, the glucose oxidase may be coated with a pH-sensitive coating. Depending on the portion of the subject's body in which device 20 is implanted, either a low (i.e., acidic), or a high (i.e., alkaline) pH may indicate infection. As described hereinabove, the glucose oxidase reduces infection, therefore, for some applications, the glucose oxidase is released in response to the pH in the vicinity of the portion in which the device is implanted.

For some applications, the pH-sensitive coating dissolves and releases the glucose oxidase in response to the pH of the vicinity passing a threshold. For some applications, the coating is configured such that the glucose oxidase is released in response to the pH of the vicinity passing a starting threshold in a first direction, and the release of the glucose oxidase is stopped in response to the pH passing a stopping threshold in a second direction opposite to the first direction. In accordance with respective applications, the starting and stopping thresholds are the same threshold, or are different from each other.

For some applications, device 20 releases the glucose oxidase slowly. For example, the device may release the glucose oxidase over a period of more than a week, more than a month, or more than six months. For some applications, the device includes a reservoir in which the glucose oxidase is stored, and from which the glucose oxidase is slowly released. For example, an artificial heart valve may include a reservoir that releases glucose oxidase over the course of one month.

For some applications, device 20 is a device that facilitates bone generation in the region of the subject's body in which the device is implanted. For example, the device may include bone graft, bone graft powder, a paste, an injected material, and/or a metal. Alternatively or additionally, the device may be a membrane, e.g., a collagen barrier or a membrane that contains a polymer, such as, expanded polytetrafluoroethylene (ePTFE) or polytetrafluoroethylene (PTFE) (marketed as Gore-Tex®). For some applications, the glucose oxidase facilitates the bone generation, for example, by reacting with native glucose that is present at the implantation site.

Reference is now made to FIG. 2, which is a schematic illustration of medical device 20 having coupled thereto particles of glucose oxidase 22, in accordance with some applications of the present invention. Device 20 is generally as described hereinabove. For some applications, the particles are particles of glucose oxidase 22. Alternatively or additionally, the particles, which are shown coupled to device 20, contain glucose oxidase.

For some applications, nanoparticles (of glucose oxidase or that contain glucose oxidase) are coupled to device 20. The glucose oxidase typically reacts with native glucose that is present in the portion of the subject's body in which device 20 is implanted, as described hereinabove. Typically, using nanoparticles of glucose oxidase, or nanoparticles that contain glucose oxidase, increases a surface area of the glucose oxidase that comes into contact with the glucose.

For some applications, device 20 is implanted within the subject's body. Subsequently (for example, in response to detecting that there is insufficient glucose oxidase coupled to the device), particles (as described hereinabove) are administered to the subject. For example, a solution that contains the particles may be injected into the subject's blood stream. The particles are attracted to the device, and become coupled to the device.

For some applications, the particles contain ligands, and device 20 comprises receptors possessing affinity for the ligand. Alternatively, the device comprises ligands and each of the particles contains a receptor possessing affinity for the ligands. For some applications, each of the particles comprises a magnetic material, and the medical device comprises a magnetic material. For some applications, the medical device comprises an antigen, and the particles each contain an antibody that couples to the antigen of the medical device. Alternatively, the medical device comprises antibodies, and each of the particles comprises an antigen that couples to the antibodies of the medical device.

It is noted that although applications have been described according to which the glucose oxidase reacts with native glucose, for some applications, device 20 has glucose coupled thereto. Alternatively, glucose is administered to the subject. The glucose oxidase particles (or the particles that contain the glucose oxidase) are administered to the subject, and react with the glucose that is coupled to device 20, and/or that is administered to the subject.

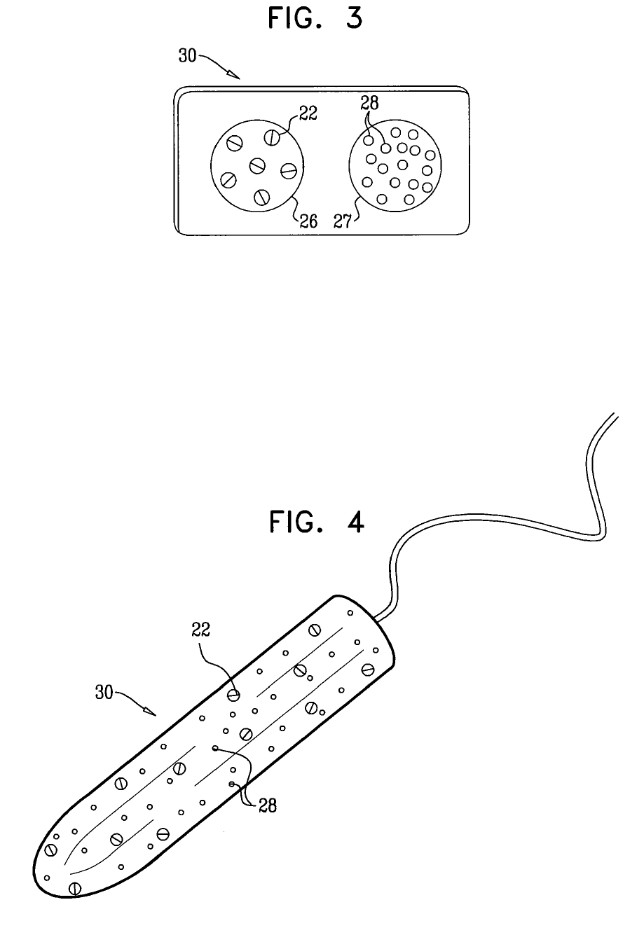

Reference is now made to FIG. 3, which is a schematic illustration of a medical element 30 (e.g., a piece of chewing gum, as shown, or a capsule, as described hereinbelow) that contains, in separate chambers 26 and 27, glucose oxidase 22 and glucose 28, in accordance with some applications of the present invention. Typically, by placing the gum in his/her mouth and chewing the gum, the subject brings the glucose and glucose oxidase into direct contact with each other, causing the glucose and glucose oxidase to react as described hereinabove.

Reference is now made to FIG. 4, which is a schematic illustration of medical element 30 (e.g., a tampon, as shown) containing dry particles of glucose oxidase 22 and glucose 28, in accordance with some applications of the present invention. Medical element 30 is typically an absorbent article that is configured to come into contact with bodily fluids, such as urine, blood, or vaginal fluid. For example, element 30 may be a diaper, a sanitary towel, a bandage, and/or other medical elements that are configured to come into contact with bodily fluids as are known in the art. Typically, the element, by coming into contact with the bodily fluid, facilitates a reaction of the glucose oxidase with the glucose by bringing the glucose and glucose oxidase in direct contact with each other. The reaction of the glucose oxidase and the glucose reduces infection as described hereinabove. For some applications, toxic shock syndrome is treated or prevented, using element 30. Alternatively or additionally an odor is prevented or reduced, using element 30.

It is noted that for some applications; medical element 30 does not contain, or have coupled thereto, a substantial amount of glucose. For example, the element may not have any glucose contained therein, or coupled thereto. For some applications, an absorbent article that is configured to come into contact with bodily fluids, as described hereinabove, contains glucose oxidase but does not contain (or have coupled thereto) glucose, or contains glucose oxidase but does not contain (or have coupled thereto) a substantial amount of glucose. The glucose oxidase reacts with native glucose that is present in a bodily fluid of a subject (such as urine, blood, or vaginal fluid) when the article comes into contact with the bodily fluid.

For some applications, a device, or a substance that contains glucose oxidase is administered to a subject's intestine. For example, the device may be a capsule, and the substance may be a gel, a liquid, a syrup, a powder, and/or a substance that is administered endoscopically to the intestine (e.g., by spraying the substance into the intestine from the distal end of an endoscope.)

For some applications, the substance does not contain glucose having a mass thereof that exceeds 0.01 percent of the mass of the glucose oxidase. For some applications, the substance reduces a presence of glucose in the intestine by facilitating a reaction of the glucose oxidase with native glucose that is present in the intestine. For example, the substance may be administered to an obese or to a diabetic subject. Alternatively or additionally, the substance reduces absorption of the intestine by facilitating a reaction of the glucose oxidase with native glucose that is present in the intestine. Further alternatively or additionally, the substance reduces inflammation in the intestine by facilitating a reaction of the glucose oxidase with native glucose that is present in the intestine. For example, the substance may be administered to a subject suffering from inflammatory bowel disease. For some applications, the substance reduces infection in the intestine, and/or reduces residues that are bound to the surface of the intestine.

For some applications, the substance contains glucose, and the substance performs the above functions by facilitating a reaction between the glucose oxidase and the glucose, while the substance is inside the intestine.

For some applications, a capsule is administered to the subject, and glucose oxidase is disposed inside the capsule. For some applications, the capsule has an environmentally-sensitive coating, e.g., a pH-sensitive coating. The coating is configured to dissolve in a given portion of the subject's gastrointestinal tract, for example, in the subject's stomach, duodenum, or colon. Such coatings are described, for example, in US 2006/0276844 to Alon, which is incorporated herein by reference.

For some applications, in response to the dissolution of the capsule coating, the glucose oxidase is released into the portion of the gastrointestinal tract, and reacts with native glucose of the gastrointestinal tract. For some applications, the reaction of the glucose oxidase with the native glucose reduces infection in the portion of the gastrointestinal tract in which the capsule coating dissolves. Alternatively or additionally, the capsule contains glucose, in addition to the glucose oxidase. For example, as described hereinabove with reference to FIG. 3, the capsule may include a first chamber that contains the glucose, and a second chamber that contains the glucose oxidase. In response to the dissolution of the capsule coating, the glucose oxidase comes into contact with the glucose, and reacts with the glucose, as described hereinabove.

For some applications, tissue of the subject's gastrointestinal tract substantially does not come into contact with the glucose oxidase of the capsule. For example, the capsule coating may be permeable with respect to gastrointestinal fluids of the subject, but substantially impermeable with respect to glucose oxidase. Gastrointestinal fluids enter the capsule and facilitate a reaction between glucose oxidase and glucose (for example, native glucose of the gastrointestinal fluids, or glucose that is contained within the capsule). The reaction of the glucose oxidase and the glucose releases hydrogen peroxide from the capsule into the gastrointestinal tract. However, substantially no glucose oxidase is released from the capsule into the gastrointestinal tract. For some applications, the substantial lack of contact between the glucose oxidase and the gastrointestinal tract tissue reduces the risk of an allergic reaction to the glucose oxidase, by the subject.

For some applications, glucose oxidase is administered to a subject, in order to treat a surgical site infection of the subject resulting from bowel surgery. For example, a capsule that has a coating that is configured to dissolve in the subject's colon may be provided. The capsule is administered to a subject suffering from surgical site infection as a result of colon surgery. For some applications, such a capsule is administered to the subject prophylactically. For example, the capsule may be administered to a subject who is going to undergo bowel surgery, several days (e.g., one to seven days) before the commencement of the surgery. For some applications, glucose oxidase is administered to the subject to treat surgical site infection, by administering to the subject a capsule, as described hereinabove. Alternatively or additionally, the glucose oxidase is administered to the subject, in accordance with one or more of the other techniques described hereinabove.

For some applications, glucose oxidase is administered to a subject in order to treat a condition of the subject's gastrointestinal tract, such as a bowel infection, traveler's diarrhea, inflammatory bowel syndrome, Crohn's disease, ulcerative colitis, diarrhea and other symptoms associated with clostridium difficile, symptoms associated with helicobacter pylori, and/or colitis. For some applications, the glucose oxidase is administered as a prophylactic treatment for one or more of the aforementioned conditions. For example, glucose oxidase is administered to a subject before, during, and/or after the subject travels, as a prophylactic treatment for traveler's diarrhea. For some applications, glucose oxidase is administered to the subject to treat the aforementioned conditions, by administering to the subject a capsule, as described hereinabove. Alternatively or additionally, the glucose oxidase is administered to the subject, in accordance with one or more of the other techniques described hereinabove.

For some applications, a powder composition (e.g. talcum powder), in dry form, includes glucose oxidase and glucose. Typically, the powder, by contacting a bodily fluid of the subject, generates hydrogen peroxide by facilitating a reaction of the glucose oxidase with the glucose. For example, the powder contacts sweat on the subject's skin, by being applied to the subject's skin, thereby reducing an odor of the subject's skin.

For some applications, a powder composition, in dry form, that includes glucose oxidase and glucose is configured to be added to a hair dye solution. For example, a user adds the powder to the hair dye solution within an hour prior to applying the dye solution to his/her hair.

For some applications, glucose oxidase is administered to a portion of a subject's body in order to reduce blood clotting of the portion (for example, by dissolving blood clots that are present in the portion). Typically, the glucose oxidase reduces the level of clotting by reacting with native glucose that is present in the portion. For some applications, the glucose oxidase is administered to a subject who suffers, generally, from hyper-coagulation. For some applications, the glucose oxidase is administered to a portion of the subject's body that is in a hyper-coagulation state, for example, a portion in which a stent is implanted.

For some applications, glucose oxidase is administered into a portion of a subject's body that is suffering from local infection. For example, the glucose oxidase may be administered to a pimple, an area of dental infection (e.g., a periodontal pocket), or to a region of periapical infection. The glucose oxidase reduces infection in the portion by facilitating a reaction of the glucose oxidase with native glucose that is present in tissue of the subject in the portion. For example, a solid pellet of glucose oxidase, or a pellet that contains glucose oxidase may be inserted into the portion. For some applications, the pellet has a volume of 0.01 cc-0.1 cc, for example, in order to be sized appropriately for insertion into a pimple. For some applications, the pellet also contains glucose and the pellet reduces infection in the portion by contacting a fluid in the portion, which facilitates a reaction of the glucose oxidase with the glucose. For some applications, a solution that contains glucose oxidase (and, optionally, glucose) is injected into the portion. Alternatively, a needle is inserted into the portion, glucose oxidase (and, optionally, glucose) being disposed on an outer surface of the needle.

For some applications, a pellet that has dimensions that are similar to those of the PerioChip® (i.e., 4 mm by 5 mm by 0.35 mm) contains glucose oxidase. The pellet is inserted into an area of gingival infection (or that is prone to gingival infection), such as a periodontal pocket. For some applications, the pellet additionally contains chlorhexidine gluconate, and/or another anti-microbial agent.

For some applications, a varnish containing glucose oxidase is applied to a subject's body. For example, a nail varnish containing glucose oxidase may be applied to nails of a subject who suffers from onychomycosis, in order to reduce the infection, as described hereinabove. In accordance with respective applications, glucose oxidase is included in a varnish that is applied to bone, cartilage, or skin.

For some application, glucose oxidase is used for water purification. For example, a capsule that includes glucose oxidase and glucose may be placed in water that is suspected of containing bacteria. For some applications, before the capsule dissolves in the water, the glucose oxidase and the glucose are contained within separate chambers within the capsule, for example, generally as described with reference to FIG. 3. Alternatively, the glucose oxidase and the glucose are both in solid particulate form within the capsule, before the dissolution of the capsule in the water. For some applications, the capsule contains additional ingredients, such as vitamins, and/or flavorings. For some applications, the capsule is an effervescent capsule.

For some applications, the water purification capsule includes a coating that is permeable with respect to water, but substantially impermeable with respect to glucose oxidase. Water enters the capsule and facilitates a reaction between the glucose oxidase and the glucose, thereby releasing hydrogen peroxide from the capsule into the water. However, substantially no glucose oxidase is released from the capsule into the water.

For some applications, an element (e.g., a filter) that is coated with glucose oxidase is placed inside a blood vessel of a subject. The glucose oxidase causes the release of hydrogen peroxide into the subject's blood, by reacting with native glucose in the subject's blood. For some applications, the filter treats a bacterial infection of the subject's blood, by causing the release of the hydrogen peroxide. For some applications, the release of hydrogen peroxide into the subject's blood in the described manner is slower and more controlled than if hydrogen peroxide were directly administered into the subject's bloodstream. For some applications, techniques as described herein (for example, the use of a selectively permeable coating) are used to prevent the release of glucose oxidase from the element into the subject's blood.

Alternatively or additionally, the subject's blood is removed from the subject's body, and is passed through an extracorporeal glucose oxidase filter. As described hereinabove, the filter treats a bacterial infection of the subject's blood by causing the release of the hydrogen peroxide. Subsequently, the subject's blood is passed back into the subject's body. For some applications, the extracorporeal glucose oxidase filter exchanges blood out with the subject's body in a similar manner to dialysis machines.

The following experiments were conducted to test the effect of various parameters of GOx activity:

Experiment 1: Effect of GOx Concentration on GOx Activity

An assay was set up that included:
48 mM sodium acetate buffer, pH 5.1
0.16 mM o-Dianisidine
1.61% (w/v) glucose
2 units/ml peroxidase

The above components undergo the following reactions in combination with GOx:

β-D-glucose+O2+H2O→D-Gluconolactone+H2O2 Reaction 1

H2O2+o-Dianisidine(reduced)→o-Dianisidine(oxidized) Reaction 2

Reaction 1 is catalyzed by the GOx, and Reaction 2 is catalyzed by peroxidase.

Different concentrations of GOx were added to the assay and the optical density of the assay after five minutes was measured with 490 nm light. The optical density of the assay at 490 nm indicates the concentration of oxidized o-Dianisidine in the assay, oxidized o-Dianisidine being the final product of Reactions 1 and 2. Thus the optical density of the assay at 490 nm is indicative of the activity of the GOx, ceteris paribus. The assay was set up in a 96-well plate, in accordance with enzyme-linked immunosorbent assay (ELISA) techniques, as are known in the art. An ELISA reader, as is known in the art, was used to determine the optical density of the assay.

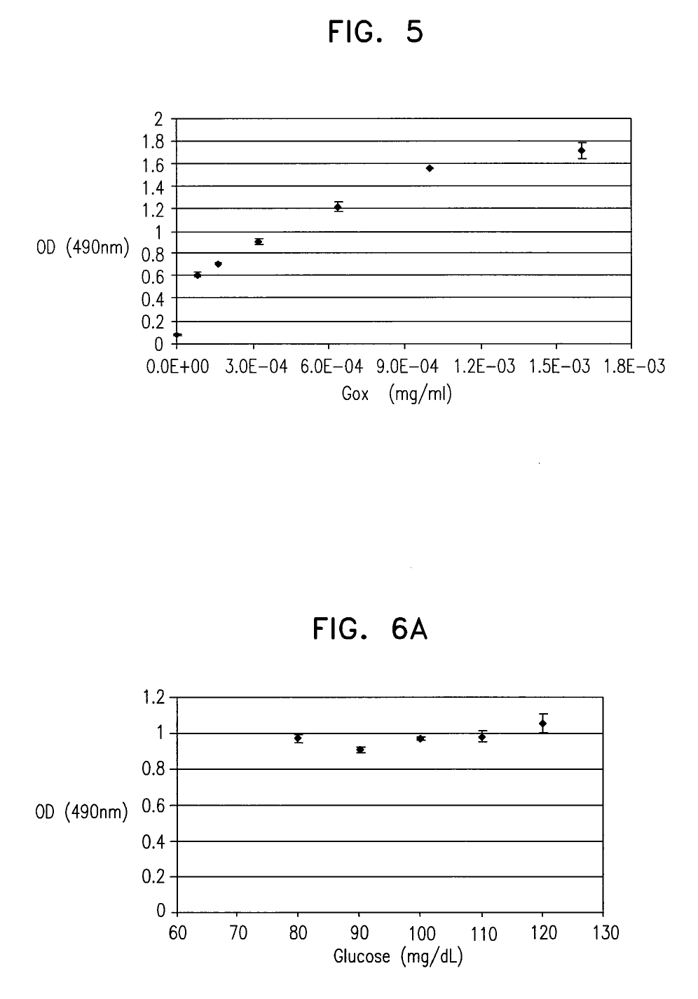

Reference is now made to FIG. 5, which is a graph showing the results of Experiment 1. It may be observed that there is a linear relationship between the concentration of the GOx and the optical density of the assay at 490 nm, for GOx concentrations of between 0.08 μg/ml and 1 μg/ml. At concentrations of greater than 1 μg/ml, the effect of the concentration of the GOx on the optical density of the assay is less. This indicates that in order to increase GOx activity it may be effective to increase GOx concentration up until a concentration of about 1 μg/ml. However, increasing the concentration of the GOx above 1 μg/ml may have a more limited effect (but not necessarily a trivial effect).

Experiment 2: Effect of Glucose Concentration on GOx Activity

An assay was set up, in accordance with the techniques described with reference to Experiment 1. The assay included:
48 mM sodium acetate buffer, pH 5.1
0.16 mM o-Dianisidine
0.03 mg/ml GOx
2 units/ml peroxidase

The above components, in combination with glucose, undergo Reactions 1 and 2 described with reference to Experiment 1 hereinabove.

Different concentrations of glucose were added to the assay, and the optical density of the assay after five minutes was measured with 490 nm light.

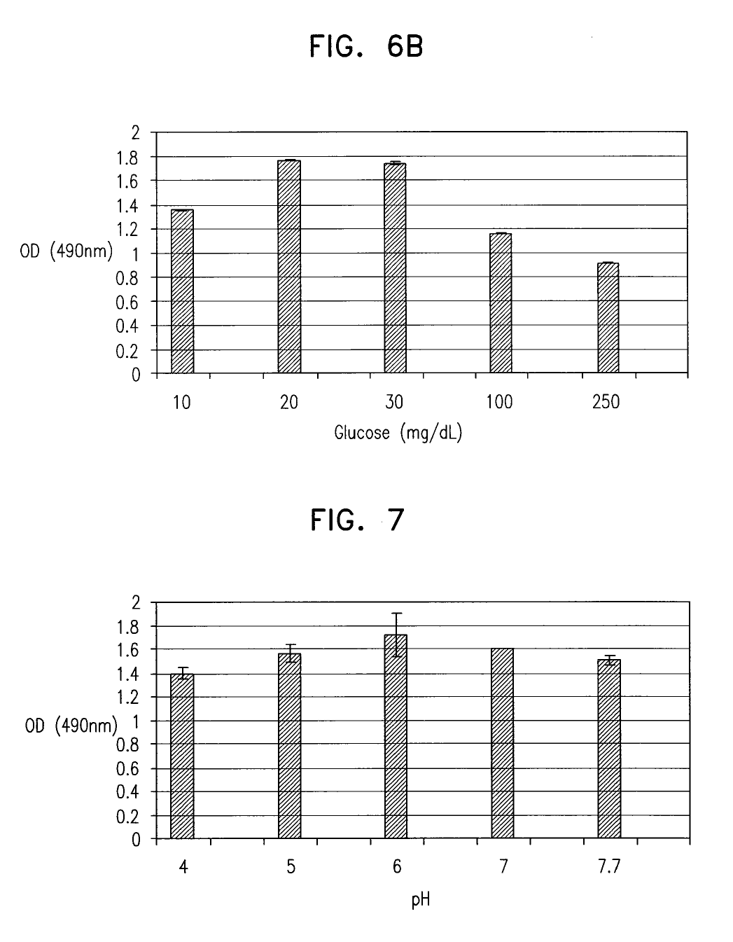

Reference is now made to FIG. 6A, which is a graph showing a first set of results for Experiment 2. It may be observed that GOx at 0.03 mg/ml concentration is active at glucose levels of 80-120 mg/dL. This indicates that GOx could be active when combined with native glucose in a subject's blood. Dependency on glucose concentration within the aforementioned range is minor. Therefore based on the results shown in FIG. 6A, it can be assumed that GOx has approximately constant activity for all glucose concentrations within the range of 80-120 mg/dL.

A further set of results were collected using a similar assay but at a different time, using glucose concentrations of a wider range, i.e., 10 mg/dL to 250 mg/dL. Reference is now made to FIG. 6B, which shows the optical density of the assay at 490 nm after five minutes plotted against glucose concentration for the wider range of glucose concentrations. It may be observed that the greatest activity of the glucose oxidase is at a glucose concentration of between 10 mg/dL and 100 mg/dL, e.g., 20-30 mg/dL. It is noted that the optical density of the assay at a glucose concentration of 100 mg/dL differs between FIGS. 6A and 6B. This may be due to the laboratory conditions when the data shown in FIG. 6B were collected having been different from the laboratory conditions when the data shown in FIG. 6A were collected.

Experiment 3: Effect of pH on GOx Activity

An assay was set up, in accordance with the techniques described with reference to Experiment 1. The assay included:
0.16 mM o-Dianisidine dissolved in sodium acetate buffer
1.61% (w/v) glucose dissolved in sodium acetate buffer
2 units/ml peroxidase
0.03 mg/ml GOx dissolved in sodium acetate buffer

The above assay was set up with sodium acetate buffer of a pH that varied from 4 to 7.7.

Reference is now made to FIG. 7, which shows the optical density of the assay at 490 nm after five minutes, plotted against the pH of the assay. It may be observed that, based on the results shown in FIG. 7, GOx activity is not substantially influenced by pH conditions in the assay within the aforementioned range of pH levels. Thus, based on the results shown in FIG. 7, the inventors hypothesize that the GOx enzyme can operate at least within a range of pH levels of 4 to 7.7.

The following experiment was conducted to determine the effectiveness and sustainability of GOx as an antibacterial agent.

Experiment 4: Effectiveness and Sustainability of GOx as an Antibacterial Agent

Materials and Methods.
A Versamax™ temperature-controlled spectrophotometeric plate reader was used to continuously monitor growth of bacteria. This device accepts standard microtiter plates and maintains a constant temperature as desired. It therefore serves both as an incubator and as a plate reader that quantitatively monitors bacterial growth by following the development of turbidity in the wells. The device is supported by software which allows generation of individual growth curves for each well.
Enterococcus faecalis strain G was used. This bacterium was selected to establish a baseline to which other bacteria may be compared in the future. It is a stable strain and is streptomycin-resistant, which allows the use of this antibiotic agent in the growth media to reduce the incidence of accidental contamination with other bacterial strains.
Growth medium and conditions. The tests were conducted using Brain-Heart Infusion broth (BHI) as growth medium. This medium contained 1 mg/100 ml glucose which is approximately equivalent to serum concentration of glucose. A temperature of 37 C was maintained throughout the test.
Antibacterial agents. The antibacterial agents tested in the experiment were:
   (a) GOx preparation: glucose oxidase from Aspergillus niger (Sigma, type x, G7141). The GOx was initially dissolved at a concentration of 1 mg/ml from which further dilutions were prepared, as required.
   (b) Triclosan preparation: Irgasan® (Sigma 72779) was used. The Triclosan was initially dissolved in water at a concentration of 10 mg/ml. This required a moderate heating (80 C) of the solution, since the material as provided does not readily dissolve in aqueous solution at that concentration.
Non-woven fabric. Two types of non-woven fabric were used. The material was cut to standard size of 6 mm2. Each piece of material was then attached to the surface of a microtiter-plate-insert and saturated with a test material, which was allowed to dry on the material, before being tested for antimicrobial activity.
Experimental design: Experiments were designed to test GOx and Triclosan for their antibacterial effect in terms of minimal inhibitory concentration (“MIC”) and sustained antimicrobial effect. The potential of non-woven fabric to serve as a carrier of GOx was also explored.
Tests conducted:
   (a) Determination of the MIC of GOx: The antibacterial effect of GOx was tested in 24 hour assays in order to define its MIC. The growth medium contained 1 mg/100 mL glucose, which is approximately equivalent to the concentration that may be present in human blood. It served as a substrate for the GOx to generate the oxygen species which are responsible for the antibacterial activity of GOx.
   Each well contained 200 μL of sterile growth medium to which a range of GOx concentrations was added. Bacteria were added at 106 CFU/well and the turbidity in the well was continuously monitored for up to 24 h to monitor the growth of the bacteria. Each group consisted of 8 wells with respective concentrations. Wells containing bacteria with no antibacterial agent served as a positive control. Wells with no bacteria served as a negative control.
   (b) Determination of sustained antibacterial effect of GOx: GOx was added to the experimental wells at respective concentrations, and the wells were maintained under generally similar conditions to each other. GOx was added to one of the wells at a concentration of 6 μg/well (30 μg/mL), which was established as the MIC in preceding experiments, or higher. Bacteria were added to the same wells at time 0 and then at 24 and 48 h. Bacterial growth was monitored for 24 h after each addition.
   (c) Determination of the MIC of Triclosan: The antibacterial effect of Triclosan was tested in 24 hour assays in order to define its MIC. Each well contained 200 μL of sterile growth medium. A range of Triclosan concentrations was added to respective wells. Bacteria were added at 106 CFU/well, and the turbidity in the well was continuously monitored for 24 h to monitor bacterial growth. Each group consisted of 8 wells with respective concentrations. Wells containing bacteria with no antibacterial agent served as a positive control. Wells with no bacteria served as a negative control.
   (d) Antibacterial effect of GOx attached to non-woven fabric. To test for the potential of GOx absorbed on non-woven materials to inhibit bacterial growth, standard pieces of non-woven material with a surface area of 6 mm were saturated with 10 μL of GOx solution (1 mg/mL), which was allowed to dry before testing. Each sample was attached to a special insert that allowed the material to be inserted into the wells of a microtiter plate without interfering with turbidity measurements in that well.

Results

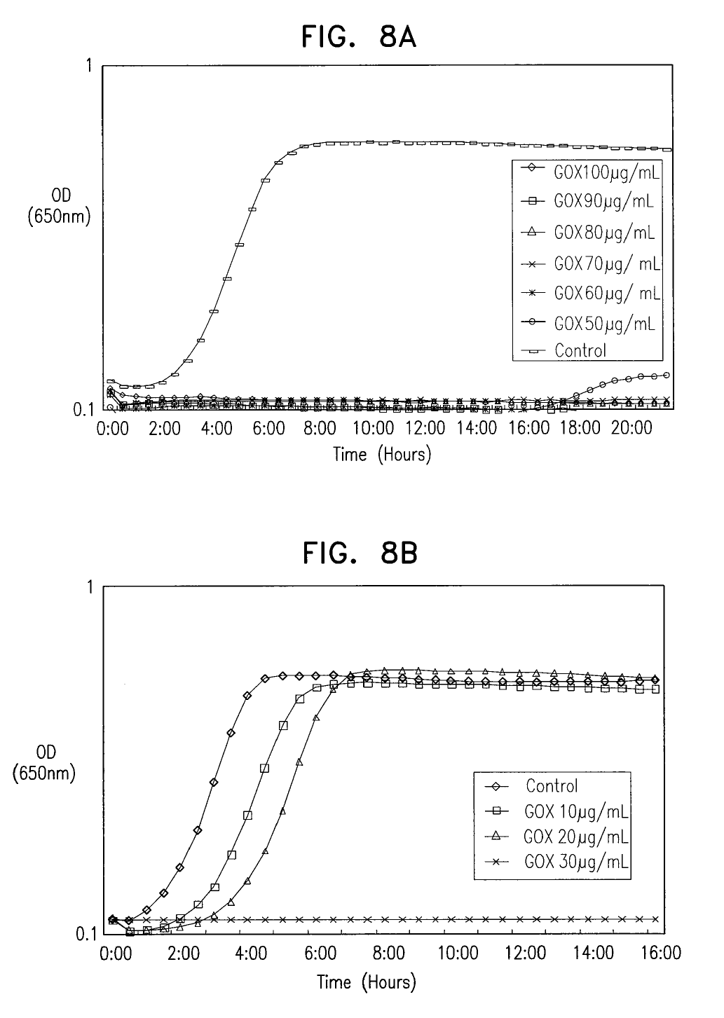

(a) MIC of GOx: Reference is made to FIGS. 8A and 8B, which include plots showing the variation of the optical density at 650 nm of the wells over 24 hours for respective concentrations of GOx. The optical density at 650 nm is indicative of the bacterial concentration of the well. It may be observed that GOx concentrations of 6 μg/well (30 μg/mL) or higher were substantially inhibitory, while a concentration of 4 μg/well (20 μg/mL) failed to inhibit the growth of the test bacterium. Based on the results shown in FIGS. 8A and 8B, the MIC of GOx for enterococcus faecalis strain G was established at 30 μg/mL.

(It is hypothesized by the inventors that the lines corresponding to GOx concentrations of 10 μg/mL, and 20 μg/mL, and the line corresponding to the control, dip between 0:00 hr and 03:00 hr due to the system calibrating itself, the starting calibration of the system having been incorrect, due to humidity of the system. It is noted that the straightness of the line corresponding to a GOx concentration of 30 μg/mL indicates zero bacterial growth.)

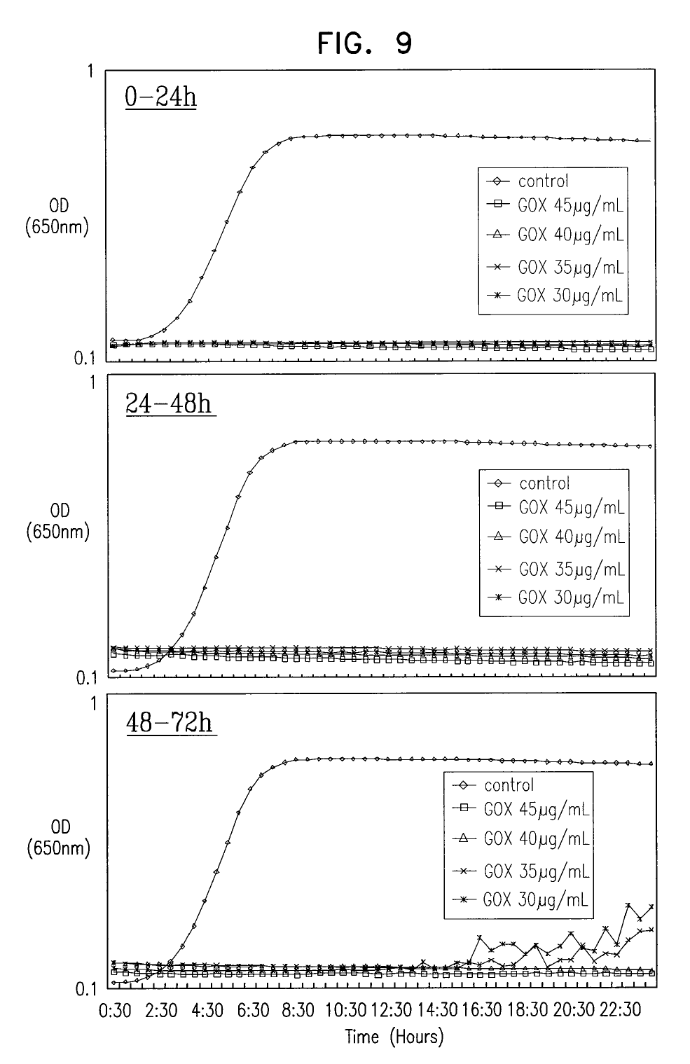

(b) Sustained antibacterial effect of GOx: Reference is made to FIG. 9, which includes plots showing the optical densities of the wells over 72 hours for respective concentrations of GOx. It may be observed that there was substantially complete inhibition of bacterial growth when using GOx concentration of 6 μg/well (30 μg/mL) or higher. This occurred for up to 60 hours of incubation of the GOx in the presence of its substrate, glucose, and its target, bacteria. After this time point “noise” appeared in the system. It is hypothesized by the inventors that the noise is due to growth medium exhaustion.

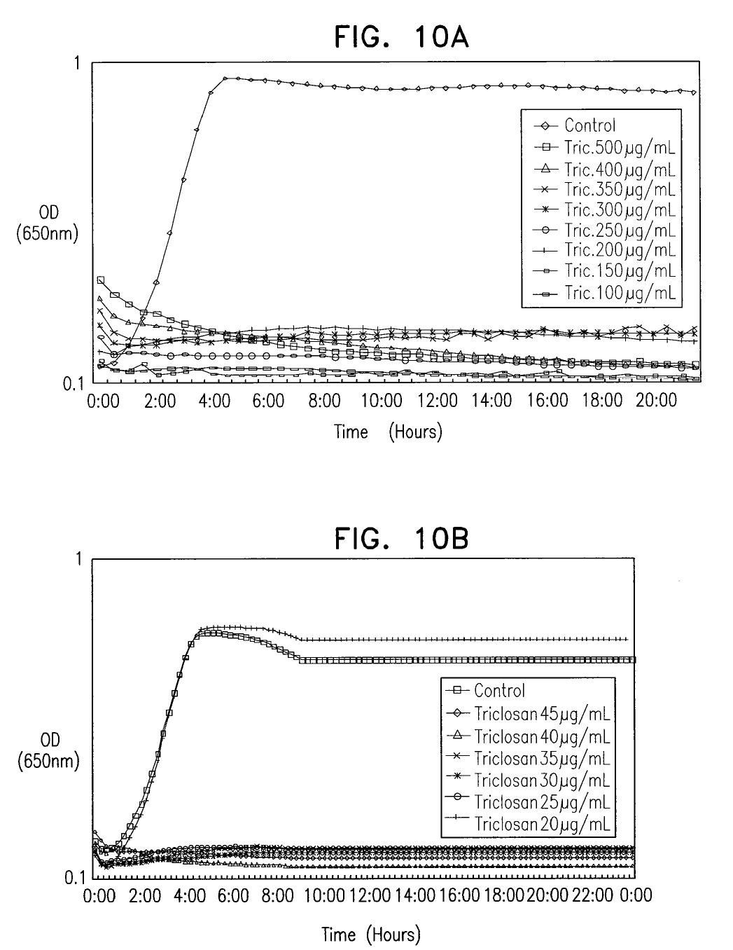

(c) MIC of Triclosan: Reference is made to FIGS. 10A and 10B, which include plots showing the variation of the optical density of the wells at 650 nm over 24 hours for respective concentrations of Triclosan. It may be observed that Triclosan concentrations of 5 μg/well (25 μg/mL) and above were substantially inhibitory, while a concentration of 4 μg/well (20 μg/mL) failed to inhibit the growth of the test bacterium. Based on the results shown in FIGS. 10A and 10B, the MIC of Triclosan for enterococcus faecalis strain G was established at 25 μg/mL. It is noted that no data are available yet regarding the sustained antibacterial effect of Triclosan.

(d) Antibacterial effect of GOx attached to non-woven fabric: The results of the four experiments conducted with the non-woven fabrics were non-conclusive. They gave erratic results most probably due to technical aspects.

### CONCLUSIONS

(a) The system used is suitable for measurement of the antibacterial effects of GOx and Triclosan.

(b) GOx has an antibacterial activity on the tested bacterium with an MIC of 30 μg/mL.

(c) GOx has a sustained antibacterial activity, at the above concentration, which was maintained and not exhausted for up to at least 60 h in cultures containing both glucose and bacteria. This indicates that GOx could be administered to a subject and configured to have an antibacterial effect, by reacting with the subject's native glucose for at least 60 hours. If the noise that developed after 60 hours was due to growth medium exhaustion, then when administered to the human body, it is possible that the antibacterial activity of GOx could be sustained for a longer period.

(d) Triclosan had an antibacterial activity on the tested bacterium with an MIC of 25 μg/mL.

(e) The experiments with GOx which was absorbed on non-woven fabric have been non-conclusive so far.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
an implantable medical device for implanting in contact with a portion of a subject's body; and
glucose oxidase, having a mass thereof, coupled to the medical device,
wherein the medical device does not have coupled thereto a mass of glucose that exceeds 0.01 percent of the mass of the glucose oxidase.

2. (canceled)

3. The apparatus according to claim 1, wherein the glucose oxidase comprises nanoparticles of glucose oxidase.

4. The apparatus according to claim 1, wherein the medical device is configured to generate hydrogen peroxide in a vicinity of the portion by facilitating a reaction of the glucose oxidase with native glucose that is present in tissue of the subject in the vicinity.

5. The apparatus according to claim 1, wherein the medical device is configured to reduce infection in a vicinity of the portion by facilitating a reaction of the glucose oxidase with native glucose that is present in tissue of the subject in the vicinity.

6. The apparatus according to claim 1, wherein the medical device comprises a suture.

7. The apparatus according to claim 1, wherein the medical device comprises a catheter.

8.-10. (canceled)

11. The apparatus according to claim 1, wherein the medical device is configured to reduce blood coagulation in a vicinity of the portion by facilitating a reaction of the glucose oxidase with native glucose that is present in tissue of the subject in the vicinity.

12. The apparatus according to claim 11, wherein the device comprises a stent.

13. The apparatus according to claim 1, wherein the medical device is configured to reduce fibrotic encapsulation of the device by facilitating a reaction of the glucose oxidase with native glucose that is present in tissue of the subject in a vicinity of the portion.

14.-18. (canceled)

19. The apparatus according to claim 1, wherein the medical device is configured to release the mass of glucose oxidase from the medical device over a period of more than a week.

20.-21. (canceled)

22. The apparatus according to claim 1, wherein the medical device comprises a heart valve configured to be implanted in a heart of the subject.

23. (canceled)

24. The apparatus according to claim 1, wherein the medical device comprises a bone generation facilitator configured to facilitate bone generation in a region of the subject's body by being placed in the region.

25.-43. (canceled)

44. A method, comprising:
providing an implantable medical device for implanting in contact with a portion of a subject's body; and
coupling to the device glucose oxidase having a mass thereof, the device not having coupled thereto glucose having a mass thereof that exceeds 0.01 percent of the mass of the glucose oxidase.

45. (canceled)

46. The method according to claim 44, wherein coupling the glucose oxidase to the device comprises coupling nanoparticles of glucose oxidase to the device.

47. The method according to claim 44, wherein coupling comprises configuring the coupling such that hydrogen peroxide is generated in a vicinity of the portion by facilitating a reaction of the glucose oxidase with native glucose that is present in tissue of the subject in the vicinity, by implanting the device in contact with the portion.

48. The method according to claim 44, wherein coupling comprises configuring the coupling such that infection is reduced in a vicinity of the portion by facilitating a reaction of the glucose oxidase with native glucose that is present in tissue of the subject in the vicinity, by implanting the device in contact with the portion.

49.-51. (canceled)

52. The method according to claim 44, wherein providing the medical device comprises providing a catheter.

53. The method according to claim 44, wherein providing the medical device comprises providing a suture.

54. The method according to claim 44, wherein coupling comprises configuring the coupling such that blood coagulation is reduced in a vicinity of the portion, by facilitating a reaction of the glucose oxidase with native glucose that is present in tissue of the subject in the vicinity, by implanting the device in contact with the portion.

55. The method according to claim 54, wherein providing the device comprises providing a stent.

56. The method according to claim 44, wherein coupling comprises configuring the coupling such that fibrotic encapsulation of the device is reduced, by facilitating a reaction of the glucose oxidase with native glucose that is present in tissue of the subject in a vicinity of the portion, by implanting the device in contact with the portion.

57.-60. (canceled)

61. The method according to claim 44, wherein coupling comprises configuring the coupling such that the mass of glucose oxidase is released from the device over a period of more than a week.

62.-63. (canceled)

64. The method according to claim 44, wherein providing the medical device comprises providing a heart valve configured to be implanted in a heart of the subject.

65. (canceled)

66. The method according to claim 44, wherein providing the medical device comprises providing a bone generation facilitator configured to facilitate bone generation in a region of the subject's body by being implanted in the region.

67.-121. (canceled)

122. A method comprising:
identifying a subject as suffering from a condition; and
in response to the identifying, providing a capsule configured to be administered to a gastrointestinal tract of a subject, the capsule including therein glucose oxidase.

123. The method according to claim 122,
wherein providing the capsule comprises providing a capsule having a pH-sensitive coating that is configured to dissolve in a given portion of the gastrointestinal tract, the glucose oxidase being contained within the capsule,
further comprising administering the glucose oxidase to the portion of the gastrointestinal tract, by dissolving the pH-sensitive coating, by administering the capsule to the subject.

124. The method according to claim 122, wherein identifying the subject as suffering from the condition comprises identifying the subject as suffering from a condition selected from the group consisting of: surgical site infection, bowel infection, traveler's diarrhea, inflammatory bowel syndrome, Crohn's disease, ulcerative colitis, a condition associated with clostridium difficile, a condition associated with helicobacter pylori, and colitis.

125. The method according to claim 124, wherein providing the capsule comprises providing the capsule in order to prophylactically treat the condition.

126.-129. (canceled)
